(19) United States
(12) Patent Application Publication
(10) Pub. No.: US 2005/0267424 A1
(43) Pub. Date: Dec. 1, 2005

(54) Wound chamber with remote access portal

(76) Inventors: Elof Eriksson, Wellesley, MA (US); W. Robert Allison, Sudbury, MA (US); Christian Baker, Weymouth, MA (US)

Correspondence Address: QUARLES & BRADY LLP, 411 E. WISCONSIN AVENUE, SUITE 2040, MILWAUKEE, WI, 53202-4497, US

(21) Appl. No.: 11/130,490

(22) Filed: May 17, 2005

Related U.S. Application Data
(60) Provisional application No. 60/571,837, filed on May 17, 2004.

(51) Int. Cl. A61F 13/00

(52) U.S. Cl. 604/304

(57) ABSTRACT

A wound dressing includes a base, a chamber, a tube, and a portal. The chamber extends from the base to define a treatment space. The tube has a first end coupled to the chamber. The tube communicates with the treatment space. The portal is coupled to a second end of the tube.

### CROSS-REFERENCE TO RELATED APPLICATIONS

This claims benefit of U.S. Provisional Patent Application No. 60/571,837 filed on May 17, 2004.

### STATEMENT REGARDING FEDERALLY SPONSORED RESEARCH OR DEVELOPMENT

Not applicable.

### BACKGROUND OF THE INVENTION

1. Field of the Invention

The invention relates generally to the field of wound treatment, and more particularly, to a wound chamber with a remote access portal.

2. Description of the Related Art

Various techniques are employed to treat open wounds. In some cases, open wounds may be treated with moist or dry gauze. However, such treatment may result in excessive pain, dehydration of the wound, loss of fluids and proteins, loss of heat or delayed healing. To delay the appearance of infection, burn wounds may be additionally treated with antibacterial creams and the like.

Open wounds appear to heal faster in an environment that is somewhere between moist and dry. Partial thickness wounds heal faster when covered with a polyethylene film than when exposed to air. Conventionally, dressings with some water permeability provide the optimal conditions for healing.

Wound chambers for protecting open wounds and providing environmental control of the treatment site have been developed. For example, an exemplary wound chamber and methods for its use are described in U.S. Pat. No. 5,152,757, entitled System for Diagnosis and Treatment of Wounds,” by Elof Eriksson, the specification of which is incorporated herein by reference in its entirety.

A wound chamber typically includes a chamber for enclosing a predetermined surface area about a wound on a patient. The wound chamber is sealed to the skin surrounding the wound. In addition to other features, the wound chamber may have a portal for introducing treatment fluid and treatment additives into the wound chamber and extracting wound fluid and/or air from the wound chamber. Such operations are typically performed using a syringe or similar delivery/extraction device. For example, the portal may be an injection portal made of a self-sealing material through which a hollow steel needle can be passed. The use of a steel needle in close proximity to the patient creates a risk of injury to the patient and also to the health care provider. An additional risk of the needle damaging the wound chamber is also present. In some clinical applications, the wound chamber itself may be covered by a secondary dressing or covering, such as gauze or an elastic bandage. In such circumstances, a portal located directly on the chamber would be covered by the secondary dressing and would be inaccessible.

The present invention is directed to overcoming, or at least reducing the effects of, one or more of the problems set forth above.

### BRIEF SUMMARY OF THE INVENTION

One aspect of the present invention is seen in a wound dressing including a base, a chamber, a tube, and a portal. The chamber extends from the base to define a treatment space. The tube has a first end coupled to the chamber. The tube communicates with the treatment space. The portal is coupled to a second end of the tube.

### BRIEF DESCRIPTION OF THE SEVERAL VIEWS OF THE DRAWINGS

The invention may be understood by reference to the following description taken in conjunction with the accompanying drawings, in which like reference numerals identify like elements, and in which:

FIG. 1 is a perspective view of a wound dressing with a tube leading from a chamber to a portal;

FIG. 2 is a sectional side elevation view of the wound dressing of FIG. 1;

FIG. 3 is a perspective view of a fitting coupled to the tube of the wound dressing of FIG. 1;

FIG. 4 is a perspective view of a portal made of self-sealing material at the end of the tube of the wound dressing of FIG. 1;

FIG. 5 is a perspective view of a collapsible tube that may be used with the wound dressing of FIG. 1;

FIG. 6 is a perspective view of a syringe with a flexible delivery tip inserted into the collapsible tube of FIG. 5; and

FIG. 7 is a perspective view of the delivery tube equipped with a fitting and an auxiliary port of self-sealing material.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

While the present invention may be embodied in any of several different forms, the present invention is described here with the understanding that the present disclosure is to be considered as setting forth an exemplification of the present invention that is not intended to limit the invention to the specific embodiment(s) illustrated. Nothing in this application is considered critical or essential to the present invention unless explicitly indicated as being “critical” or “essential”.

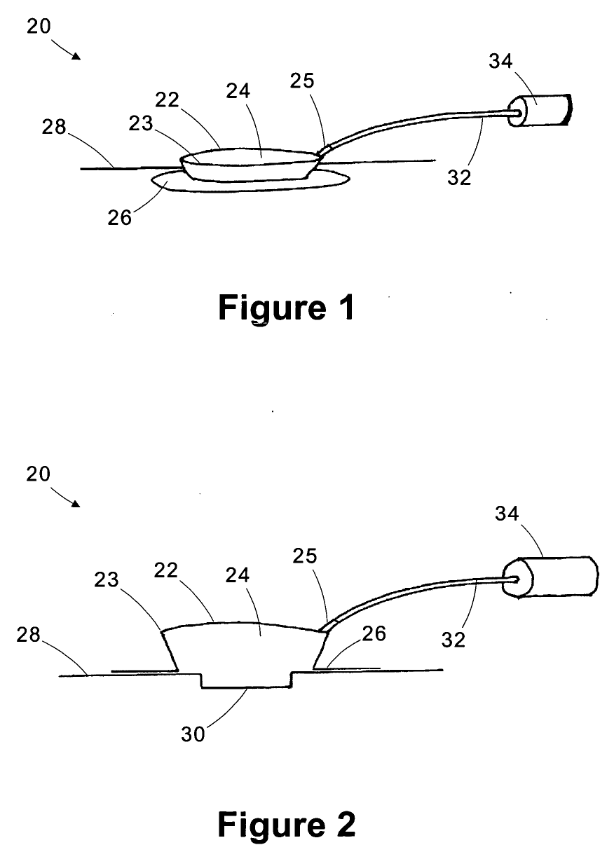

Referring first to FIGS. 1 and 2, a perspective view of a wound dressing 20 is provided. The wound dressing 20 includes a chamber 22 defining a treatment space 24 and a base 26 that may be sealed to a skin surface 28 of a patient over a wound 30. In the illustrated embodiment, the chamber 22 has a bellows configuration with a fold 23. However, the invention is not so limited, and other configurations may be used. An adhesive material may be provided on a bottom surface of the base 26 for providing a fluid tight seal with sufficient adhesive strength to prevent inadvertent removal of the wound dressing 20 or breach of the fluid tight seal during normal patient movement. Numerous adhesive materials sufficient for these purposes are known to those of ordinary skill in the art.

A tube 32 is attached to the chamber 22 and communicates with the treatment space 24. The tube 32 may be permanently fixed to the chamber 22, or a fitting 25 may be provided to allow removal of the tube 32. A distal end of the tube 32 terminates in a portal 34. In one embodiment, the portal 34 may be a female Luer fitting. As used herein the terms portal and fitting may be used interchangeably, depending on the particular implementation. A fitting generally refers to a device that mates with a complementary, interfacing device, while a portal may relate to a device into which something is inserted, such as a needle. The tube 32 may terminate at a wall of the chamber 22 as shown, or it may extend through the wall a distance and terminate within the treatment space 24. The tube 32 is sealed to the chamber 22 in such a manner as to prevent the escape of liquid or air from the treatment space. Although only one tube 32 and portal 34 are illustrated, the invention is not so limited, and multiple tubes 32 and associated portals 34 may be provided for accessing the treatment space 24. One tube 32 and portal 34 may be used as an inlet for providing treatment substances to the treatment space 24, while another tube 32 and portal 34 may be used as an outlet for removing substances from the treatment space 24. Such an inlet and outlet arrangement may be useful for administering a continuous treatment stream.

The base 26 may have various implementations. For example, the base 26 may be a cuff adapted to secure the chamber 22 to a limb. In such an embodiment, the base 26 may have a different closure device, such as a drawstring, band, strap, tab, etc., in lieu of or in combination with an adhesive material for securing the wound dressing 20 about the wound 23. The base may also be located on the portion of the body not proximate to the wound 30. For example, the wound 23 may be on the hand or forearm, while the base 26 may be attach to the body on the upper arm. A wound 23 on the scalp may necessitate the base 26 being disposed on the forehead and around to the neck.

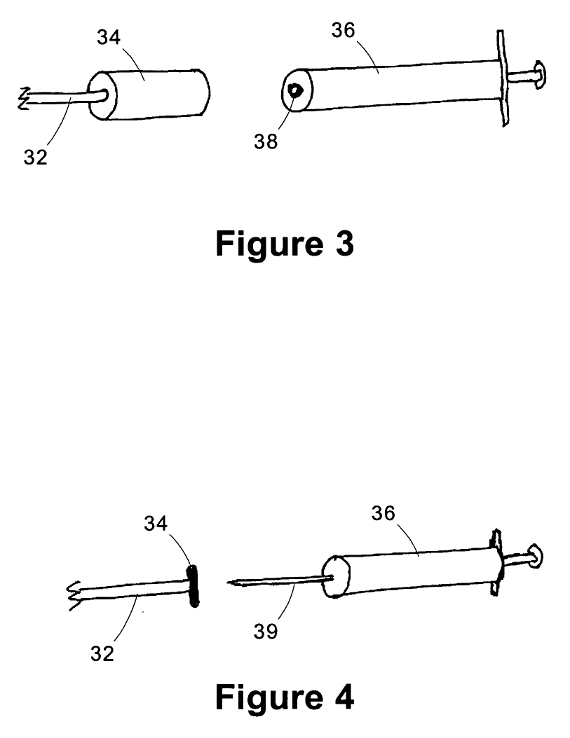

Turning now to FIG. 3, a delivery/extraction device 36, such as a syringe, may be engaged with the portal 34 to allow delivery of treatment fluid and/or treatment additives to the treatment space 24 or removal of liquid or gases from the treatment space 24. For example, the delivery/extraction device 36 may include a fitting 38, such as a male Luer fitting, complementary to a female Luer fitting used as the portal 34. In the case of complementary female and male Luer fittings, the rotation of the male fitting 38 on the delivery/extraction device 36 opens a valve in the female portal 34 extending from the tube 32. The delivery/extraction device 36 may include devices other than the syringe illustrated. For example, the delivery/extraction device 36 may include a rigid or flexible container for holding one or more liquid or powdered treatment substances that may be delivered to the treatment space 24.

The length of the tube 32 may vary with the particular implementation. In an application where the tube 32 is provided to increase the distance between the delivery/extraction device 36 to protect the patient and/or chamber 22 from a needle, the tube should be at least longer than the needle. In cases where the wound dressing 20 is intended to be covered with a secondary dressing, the tube 32 may be still longer. For example, the tube 32 may be between 1″ and 24″ in length. The tube 32 is generally flexible and may be foldable against itself depending on the specific implementation.

FIG. 4 illustrates an embodiment where the portal 34 is constructed of a self-sealing material. In this embodiment, the delivery/extraction device 36 may be a syringe having a hollow steel needle 39 that is inserted through the self-sealing portal 34 to allow injection of treatment fluids and/or treatment additives and/or withdrawal of wound fluid or air from the treatment space 24.

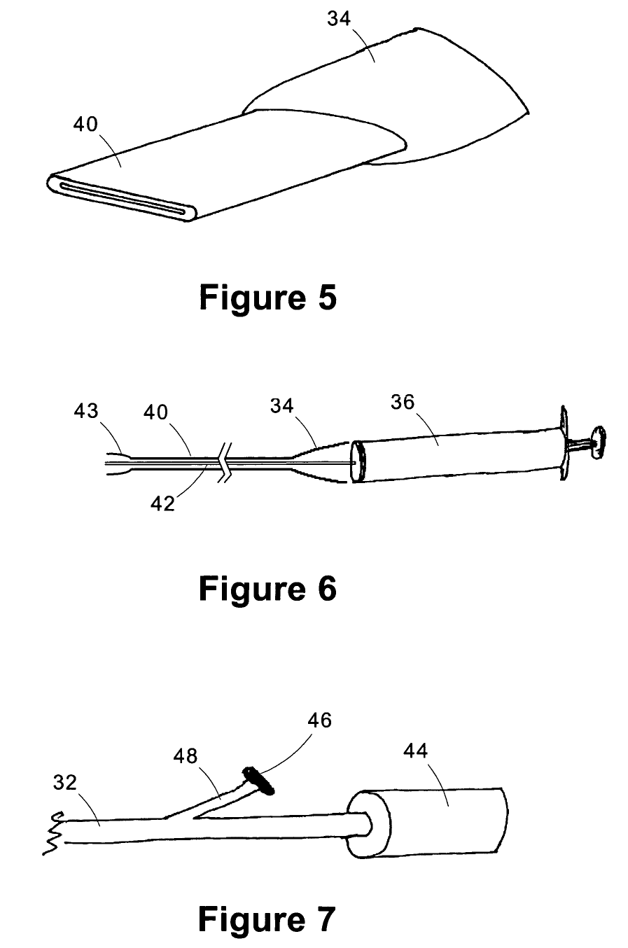

Referring to FIGS. 5 and 6, another embodiment is illustrated wherein the tube 32 of FIG. 1 is a collapsible tube 40 formed of a flexible, collapsible material that normally lies flat. The delivery/extraction device 36 may be a syringe with a flexible tip 42 that is inserted into the collapsible tube 40. The flexible tip 42 may be inserted throughout the entire length of the collapsible tube 40 such that exits the collapsible tube 40 and extends directly into the treatment space 24. In some embodiments, e.g., where fluids are being added, the flexible tip 42 may be shorter than the collapsible tube 40. The collapsible tube 40 expands as necessary to allow the insertion of the flexible tip 42 and/or treatment fluids. When the flexible tip 42 is withdrawn, the collapsible tube 40 collapses and forms a watertight and airtight seal with respect to the treatment space 24. In some embodiments, the collapsible tube 40 may have a flared end 43 where it interfaces with the chamber 22, i.e., either terminating at the chamber 22 or extending into the chamber 22. The flexible tip 42 may be positioned within the flared end 43, yet not within the treatment space 24 to allow removal of fluids from the treatment space 24.

As seen in FIG. 7, the tube 32 of FIG. 1 may be equipped with a portal 44 that is a fitting, such as female Luer fitting, as well as an auxiliary portal 46 of self-sealing material located on a branch tube 48. The availability of two portals 44, 46 increases the flexibility of the wound dressing 20 by allowing users to employ needles or needleless delivery/extraction devices 36.

The tube 32 allows the portal 34 to be located a distance away from the skin surface 28, the wound 30, and the chamber 22. Hence if treatment fluids are introduced into the treatment space 24 using a needle, the likelihood of inadvertently contacting the patient or health care provider, or breaching the chamber 22 is reduced. Also, if the wound dressing 20 is covered by a secondary dressing (not shown), the wound 30 may still be treated through the tube 32 without requiring removal of the secondary dressing.

The wound dressing 20 described above has numerous applications. The tube 32 may be used to introduce a wide variety of treatment fluids and/or additives. The wound dressing 20 allows monitoring of the wound, which is useful in enhancing the healing process. The chamber 22 allows visual monitoring of the wound 30 itself as well as the monitoring of fluid within the treatment space 24. This monitoring provides feedback to assist in the precise control of treatment variables and facilitates research. Fluid extracted from the system can be analyzed for factors that indicate wound healing status and also for the presence of deleterious factors such as microorganisms, low oxygen, high carbon dioxide and adverse pH. The fluid may be tested for the number and type of bacteria and other microorganisms per cc of fluid, the number and type of cells, the amount and type of proteins, and other factors. Clinical diagnosis of the wound physiology and the patient may also be conducted. Upon diagnosis, further treatment of the wound 30 may be commenced by introducing treatment additives and controlling treatment variables. Depending on the type of wound, the extracted fluid can be tested for: (a) the presence of microorganisms, (b) cells, (c) amount and type of protein, (d) chemicals, (e) oxygen, (f) carbon dioxide levels, and/or (g) pH. This data may be recorded and used for wound diagnosis. Once diagnosis is complete, fluid treatment intervention may be adjusted accordingly.

Additional growth factors that are produced by the wound 30 may also be measured when extracted fluid is analyzed. Additional factors that may be tested for are the presence and the amounts of various inflammatory mediators and various antigens. The presence of antigens could serve an important diagnostic purpose and may be tested with specific antibodies that would be delivered through the wound chamber. This information is useful in deciding what to replace and how to treat, and would indicate improvement of the wound 30. The wound dressing 20 establishes an environment that allows the positive factors produced by the body to be present.

An exemplary, but not exhaustive, list of treatment materials includes anesthetics, antibiotics, chemotherapeutics, growth factors, cell culture media, cells, oxygen, buffering agents, enzymes, and immune modulators. The cells added may include cells that have been genetically modified prior to transplantation in the wound 30 or may include other gene therapy additives such as DNA, genes, genetic material, genetic vectors, etc.

The wound dressing 20 also allows control of the treatment environment within the treatment space 24. An exemplary, but not exhaustive, list of treatment variables includes temperature, colloid osmotic pressure, pH, ion concentration, glucose concentration, amino acid content, fat concentration, oxygen concentration, and carbon dioxide concentration.

By controlling treatment variables and by adding selected treatment additives, a variety of treatment techniques may be implemented. Those of ordinary skill in the art, in light of this disclosure, may identify numerous such treatment techniques, and the application of the present invention is not limited to any particular treatments. By way of illustration, a non-limiting sample of treatments that may be implemented in accordance with the present invention include cell treatment techniques, such as application of stem cells, gene therapy drugs, or cellular matrix gel suspension, infection treatment techniques, such as antibacterial or bacteriostatic treatments, or general treatment techniques, such as negative pressure therapy.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

1. A wound dressing, comprising:
a base;
a chamber extending from the base to define a treatment space;
a tube having a first end coupled to the chamber, the tube communicating with the treatment space; and
a portal coupled to a second end of the tube.

2. The wound dressing of claim 1, wherein the base is attachable to a skin surface proximate to a wound, the treatment space being defined by the skin surface and the chamber.

3. The wound dressing of claim 1, wherein the tube has a circular cross section.

4. The wound dressing of claim 1, further comprising a fitting coupling the tube to the chamber.

5. The wound dressing of claim 1, wherein the portal comprises a self-sealing material.

6. The wound dressing of claim 1, wherein the portal comprises a fitting.

7. The wound dressing of claim 1, further comprising a delivery/extraction device adapted to mate with the portal.

8. The wound dressing of claim 7, wherein the portal comprises a first fitting, and the delivery/extraction device includes a second fitting complementary to the first fitting.

9. The wound dressing of claim 7, wherein the delivery/extraction device comprises a syringe.

10. The wound dressing of claim 7, wherein the tube comprises a compressible tube, and the delivery/extraction device includes a flexible tip adapted to extend into the compressible tube through the portal.

11. The wound dressing of claim 10, wherein the flexible tip is adapted to extend through the compressible tube and into the treatment space.

12. The wound dressing of claim 7, wherein the portal comprises a self-sealing material, and the delivery/extraction device includes a rigid tip adapted to penetrate the self-sealing material to communicate with the tube.

13. The wound dressing of claim 1, further comprising an auxiliary portal coupled to the tube.

14. The wound dressing of claim 1, wherein the tube comprises a first portion coupled to the chamber and the portal, and a second portion branching off the first portion.

15. The wound dressing of claim 14, further comprising an auxiliary portal coupled to the second portion of the tube.

16. The wound dressing of claim 13, wherein one of the portal and the auxiliary portal comprises a fitting and the other comprises a self-sealing material.

17. The wound dressing of claim 1, wherein the chamber has a bellows configuration including at least one fold.

18. The wound dressing of claim 1, further comprising an adhesive material applied to at least one surface of the base.

19. The wound dressing of claim 1, wherein the tube comprises a collapsible tube.

20. The wound dressing of claim 19, wherein the collapsible tube lies flat in a normal state.

21. The wound dressing of claim 19, wherein an end of the collapsible tube proximate to the chamber is flared.

22. The wound dressing of claim 1, wherein the tube comprises a first tube, the portal comprises a first portal, and the wound dressing further comprises:
a second tube having a first end coupled to the chamber, the second tube communicating with the treatment space; and
a second portal coupled to a second end of the second tube.

23. A method for treating a wound, comprising:
placing a wound dressing over the wound, the wound dressing comprising:
a base;
a chamber extending from the base to define a treatment space;
a tube having a first end coupled to the chamber, the tube communicating with the treatment space; and
a portal coupled to a second end of the tube; and accessing the treatment space through the tube.

24. The method of claim 23, wherein accessing the treatment space further comprises introducing a treatment substance into the treatment space.

25. The method of claim 24, wherein introducing the treatment substance further comprises introducing at least one of an anesthetic, an antibiotic, a chemotherapeutic, a growth factor, a cell culture medium, cells, oxygen, a buffering agent, an enzyme, an immune modulator, genetically modified cells, and a gene therapy additive.

26. The method of claim 23, wherein accessing the treatment space further comprises removing a substance from the treatment space.

27. The method of claim 26, further comprising analyzing the substance removed from the treatment space.

28. The method of claim 23, further comprising controlling a treatment variable within the treatment space.

29. The method of claim 28 wherein controlling the treatment variable further comprises controlling at least one of temperature, pressure, colloid osmotic pressure, pH, ion concentration, glucose concentration, amino acid content, fat concentration, oxygen concentration, and carbon dioxide concentration.

30. The method of claim 23, wherein accessing the treatment space further comprises implementing a treatment process comprising at least one of stem cell treatment, gene therapy drug treatment, cellular matrix gel suspension treatment, antibacterial treatment, bacteriostatic treatment, and negative pressure treatment.
